(19) United States
(12) Patent Application Publication
(10) Pub. No.: US 2009/0176294 A1
(43) Pub. Date: Jul. 9, 2009

(54) METHOD FOR THE SUBMERGED CULTIVATION OF FILAMENTOUS ORGANISMS

(76) Inventors: Michael Pescheck, Biebergemünd-Kassel (DE); Bernd Gödelmann, Frankfurt Am Main (DE); Björn-Arne Kaup, Köln (DE); Jens Schrader, Frankfurt Am Main (DE)

Correspondence Address: ROBERTS & ROBERTS, LLP;ATTORNEYS AT LAW, P.O. BOX 484, PRINCETON, NJ, 08542-0484, US

(21) Appl. No.: 12/300,824

(22) PCT Filed: May 12, 2007
(86) PCT No.: PCT/EP07/04238

§ 371 Date: Nov. 14, 2008

(30) Foreign Application Priority Data
May 16, 2006 (DE) 10 2006 022 787.5

(51) Int. Cl. C12N 1/20; C12N 1/14

(52) U.S. Cl. 435/253.6; 435/252.1; 435/254.1; 435/256.8

(57) ABSTRACT

A method for the submerged cultivation of filamentous organisms is described, where the formation of cell agglomerates, mycelial assemblages and pellets, and the adhesion to abiotic surfaces is reduced or prevented during the cultivation through the presence of particles which are insoluble or only partly soluble in the cultivation liquid and have a size of up to a few millimetres. With this method it is possible to overcome the problems hitherto in the biotechnological use of filamentous organisms.

The subject of the invention is a novel cultivation method for submerged cultures of filamentous organisms, such as fungi or bacteria, in which by the use of particles that are insoluble or only partly soluble in the nutrient broth during the cultivation process, the development of pellet-like mycelia and cell agglomerates in the course of the mycelium growth and the growth of the fungi onto abiotic surfaces is reduced or prevented.

### BACKGROUND OF THE INVENTION

Fungi are eucaryotic organisms, which because of their extensive enzyme system are used today in the most various biotechnological processes. Among others, their products are fine chemicals, antibiotics, organic acids, and enzymes (Schlee, H., Kleber, H.-P., Biotechnologie [“Biotechnology”], Gustav Fischer Verlag, Jena, 1991).

For instance, the large-scale fermentation production of citric acid and β-lactam antibiotics with fungi by submersion methods in large fermenters having a volume of up to several hundred cubic meters has already been established for years (R. D. Schmid, Taschenatlas der Biotechnologie und Gentechnik [“Pocket Atlas of Biotechnology and Gene Technology”], Wiley-VHC, Weinheim, 2002).

In recent years, the field of biotransformation has gained ever-increasing importance. In this field as well, the use of fungi is especially highly promising, because of their high metabolic potential. The large number of enzymes to be found there and of great interest in terms of preparation makes it possible to catalyze the most various reactions quite frequently chemoselectively, regioselectively, and enantioselectively, and as a result, especially in the pharmaceutical industry, to shorten the often multi-stage and sometimes highly cost-intensive chemical synthesis processes considerably. For instance, the biotransformation of steroids has manifold technical applications (Smith, L. L., Steroids, in H.-J. Rehm, G. Reed (Eds.), Biotechnology, Vol. 6a, Biotransformations, VCH, Weinheim, 31-78, 1984). This highly heterogeneous class of substances plays a decisive role above all in medicine in the production of modern antibiotics or antiinflammatory and hormonal therapeutics. Since 1952, it has been successfully possible to convert progesterone to 11α-hydroxyprogesterone using Rhizopus nigricans (Peterson, D. H., Murray, H. C., Eppstein, S. H., Reineke, L. M., Weintraub, A., Meister, P. D., Leigh, H. M., “Microbiological Transformations of Steroids: I. Introduction of Oxygen at Carbon-11 of Progesterone”, J. Am. Chem. Soc., 74, 5933-5936, 1952). For the biotransformation of steroids, there are numerous patents today that are directed to commercial processes (such as International Patent WO 97/21830, British Patent 732,686, and U.S. Pat. No. 3,294,646). Besides fungi, a series of mycelial bacteria or bacteria that grow in multi-cell assemblages play a role, for instance in the production of antibiotics. For instance, streptomycin is obtained from Streptomyces griseus, and tetracycline is obtained from Streptomyces aureofaciens. Higher fungi are also suitable as biocatalysts for the transformation of terpenoid hydrocarbons (Busmann, D., Berger, R. G., “Bioconversion of Terpenoid hydrocarbons by Basidiomycetes”, in: Maarse, H., Heij, van der, D. G. (Eds.), Trends in Flavour Research: Proceedings of the 7th Weurman Flavour Research Symposium, Noordwijkerhout, June 1993, Elsevier Science Publishers, Amsterdam, 503-507, 1994. They are oxyfunctionalized for instance into high-quality natural flavorings and fragrances for the food and cosmetics industries (Schrader, J., Berger, R. G., “Biotechnological Production of Terpenoid Flavor and Fragrance Compounds”, in: Rehm, H.-J., Reed, G. (Eds.), Biotechnology, Wiley, Weinheim, 374-422, 2001).

However, the high biotechnological potential of higher fungi is hindered by considerable difficulties in their cultivation. These problems are due to mycelium morphologies that are dependent on organism and cultivation parameters and are highly heterogeneous, resulting in major challenges for the process engineering. Also, some kinds of bacteria, such as Streptomyces, grow by developing a mycelium and in the course of fermentation tend to clump.

Because of often extremely high viscosities, the Newtonian and rheological properties of the fermentation broth are lost in the course of the cultivation. Often associated with this is thus the adhesion of the fungi, which is independent of the type of mixture and of the intensity, to inert surfaces such as reactor fixtures and measuring devices such as oxygen or pH electrodes. In some types of fungi, above all in fermentation processes involving high gasification rates, the development of air mycelia is often observed, which grow to fill the air chamber of the reactor completely. These phenomena make the mixing in the reactor more difficult and thus have a direct influence on mass transfer. Moreover, they are a hindrance to precise, replicable process control. In addition, in submerged cultures of fungi, growth in pellet form often occurs, and the shape and size of the various pellets are in turn highly variable. One process parameter that affects the pellet morphology is the rotary speed of the agitator in the bioreactor. Depending on the organism used, the resultant mycelia change their shape down to relatively small pellets, or conversely, they become either more compact or form large clumps (Fujita, M., Iwahori, K., Tatsuta, S., Yamakawa, K., “Analysis of pellet formation of Aspergillus niger based on shear stress”, J. Ferment. Bioeng. 78, 368-373, 1994). Some fungi can also increase their adhesion capability as a result of the increase in the rotary speed, and in some cases, even productivity losses have been observed. The cause of this is suspected to be the increase in the shear forces in the reactor (Gibbs, P. A., Seviour, R. J., Schmid, F., “Growth of Filamentous Fungi in Submerged Culture: Problems and Possible Solutions”, Critical Reviews in Biotechnology, 20 (1), 17-45, 2000).

Moreover, the pellet hyphenes of most higher fungi exhibit only a terminal growth. Therefore of the entire mycelium mass, only a slight portion is metabolically active. A further disadvantage of the nonuniform morphology is the fact that the supply of oxygen and nutrients to the fungi pellets, above all at relatively high biomass concentrations, is severely restricted or suppressed entirely (Huang, N. Y., Bungay, H. R., “Microprobe Measurements of Oxygen Concentrations in Mycelial Pellets”, Biotechnology and Bioengineering, Vol. 15, 1973). In the course of cultivation, the death of the internal pellet cells therefore occurs, causing a further loss of metabolically active biomass. In the case of adhering mycelia as well, similarly to the situation with pellets, the oxygen and substrate supply is difficult, so that once again only the cells at the surface are active. The consequences of these cultivation phenomena are the low productivities, compared to procaryotic biological processes, under similar process conditions.

### SUMMARY OF THE METHOD OF THE INVENTION

It has now been discovered for the first time that in the submerged cultivation of filamentous organisms in the presence of particles that are insoluble or only partly soluble in the nutrient broth and that have a size of up to several millimeters and preferably comprise metal, metal oxide, glass, plastic, carbon, crystalline salt, or a semimetal, or arbitrary mixtures of these materials, the development of cell agglomerates, mycelial assemblages and pellets as well as the tendency to adhere to abiotic surfaces during the cultivation are reduced or prevented. Thus the problems described above in the conventional submerged cultivation of filamentous organisms can be effectively reduced or suppressed by the application of the novel cultivation method according to the invention for fungi and bacteria.

The surprising effect of the particles according to the invention on the growth behavior of filamentous organisms is also notable because in bioprocesses until now, particles have been used only either for the decomposition of cells or for the sake of the targeted overgrowth of organisms (immobilization).

The decisive advantage that is attained when using the novel method is the assurance of a uniform, homogeneous morphology of the organism during the cultivation. The organism does not grow in the usual way in the form of cell agglomerates, mycelial assemblages or pellets, but instead grows homogeneously in single cells or hyphenes or in small hyphene assemblages.

The method of the invention is advantageously suitable for the submerged cultivation of filamentous fungi. This method has proved to be especially advantageous for the cultivation of the fungus of the Caldariomyces fumago type. The method of the invention can furthermore be used for the submerged cultivation of other fungi as well, or also of mycelial bacteria or bacteria that grow in multi-cell assemblages, for instance of the genus Streptomyces.

For the method according to the invention, particles that are insoluble or only partly soluble in the nutrient broth are used that preferably comprise the aforementioned materials. The possibility also exists of using silicate particles or silicate splinters (glass). It has proved especially effective to add particles comprising aluminum oxide; concentrations of 0.75% to 1.25% (w/v) in the culture medium have proven especially effective. However, still other particle concentrations can also be employed. For instance, microscopic images of the growth of the fungus Caldariomyces fumago in submerged culture when the method of the invention is employed show the avoidance of the pellets that conventionally occur (see FIGS. 1 and 2). The particles comprising aluminum oxide that are employed cause the development of the fungus mycelium in the form of single hyphenes. Besides aluminum oxide, still other materials have been successfully tested as an equivalent for use as particles according to the invention.

Although filamentous fungi or bacteria have an extensive potential for the extraction of biotechnologically relevant products, their cultivation until now involved many problems. By using the method of the invention, the possibility now exists of cultivating this filamentous fungi or mycelial bacteria or bacteria that grow in multi-cell assemblages in submerged fashion for biotechnological production processes in all forms of bioreactors or glass vessels. Since in the presence of the particles according to the invention, the cells are present in substantially smaller cell assemblage or in virtually separate form, a development of dead fungal mycelium, as could be found in the interior of the pellets in conventional submerged fermentation, does not occur.

Consequently, higher productivity of the biomass can be observed, since there is a better supply of substrates and oxygen to the cell. Thus the undersupply as well as the dieoff of cells and hence the loss of biocatalyst, as is the case in the interior of pellets, during fermentation is prevented in the presence of particles in accordance with the method presented here. A further advantage of the novel method is the lower viscosity of the cultivation broth. The presence of single hyphenes ranging to individual cells has a positive effect on the rheological properties of the culture medium. What should be stressed in particular is an improvement in the supply of the submersion-cultivated fungus or bacterium with oxygen and substrates, since thus the mass transfer properties of the fermentation broth are improved considerably. Thus the method described here represents substantial progress for the submerged cultivation of filamentous organisms for the sake of the biotechnological extraction of proteins, antibiotics, vitamins, or other compounds.

The aforementioned morphological disadvantages of filamentous fungi and bacteria have the same effect on biocatalytic processes in which cells in repose are incubated. In those methods, conventionally cultivated biomass was until now usually used afterward in a medium that does not promote growth, such as aqueous buffer, organic solvent, or a two-phase system, for the intentional biotransformation of educt molecules. In such incubations, further growth of the organisms is restricted or even unwanted; instead, a metabolic capacity that catalyzes the desired reaction is stimulated by metering the educt molecule and by the choice of suitable reaction conditions. The method of the invention can now be used for the cultivation of the filamentous fungi and mycelial bacteria or bacteria that grow in multi-cell assemblages, for conducting ensuing biocatalytic processes with cells that do not grow or grow only poorly. For the efficiency of such processes in biotransformation, in which processes cells that do not grow or grow only poorly are incubated, the finely distributed biomass resulting from the method of the invention is likewise highly advantageous.

The method of the invention makes it possible for the first time to cultivate the filamentous fungi or mycelial bacteria or bacteria that grow in multi-cell assemblages for the purpose of high throughput analysis, preferably in microtiter plate formats. Such cultivation includes the culturing of the organisms in various small volumes of nutrient medium, and as a result in a short time, various variants of organisms can be tested in parallel simultaneously in large numbers with regard to their metabolic activity. Hence principles for broad testing and optimization of cultivation conditions are possible as well.

In order to change the properties of cultivated organisms in a targeted way, it is often necessary to modify the genetic outfitting of the applicable organisms. The technologies often used for this purpose, that is, transformation or transfection, are based on the incorporation of DNA that codes for the desired properties. Moreover, with the RNA interference (RNAi) technique, the possibility exists, via the incorporation of regulator RNAs, of changing certain properties of organisms in a targeted way. Until now, filamentous organisms were only poorly accessible to such methods. In the conventional method, the mycelium must first be destroyed mechanically, which causes a considerable loss of active biomass and thus greatly reduces the transformation efficiency, for instance. By separating the hyphenes to the extent of single cells, a simplified possibility of genetic manipulation of these organisms is also attained. With the method of the invention, for the first time the genetic manipulation of filamentous fungi or mycelial bacteria or bacteria that grow in multi-cell assemblages by means of foreign or endogenous nucleic acid is made possible, or facilitated, and the efficiency is enhanced. The novel cultivation method presented here has the effect that the molecular biological transformation of these organisms by means of DNA is simplified definitively. In the cultivation method developed here, the mycelium formation is suppressed, and the cells present are for the most part vital. Therefore a drastic increase in the transformation efficiency can be assumed, which represents a decisive parameter in molecular biological tasks described above. With the thus-obtained possibility of genetic manipulation, an important prerequisite for the further biotechnological application of filamentous fungi and bacteria is created.

### COMMERCIAL APPLICATIONS

The application of the method of the invention brings about an improvement in cultivation conditions for filamentous fungi and bacteria. These organisms play a significant role in industrial biotechnological production of enzymes, antibiotics, vitamins, and other compounds, such as the types of the genuses Rhizopus (a series of steroids), Aspergillus (citric acid, gluconic acid, itaconic acid, lipase, cellulase, lactase, glucoamylase, etc.), Streptomyces (streptomycin), Penicillium (dextranase, penicillin, griseofulvin, etc.), Trichoderma (dextranase, cellulase, etc.), Fusarium (gibberelline, zearalenes, etc.) or Cephalosporium (cephalosporine). The use of the particles described here during such fermentation processes can increase the productivity of these processes considerably, since for a desired product formation, a higher proportion of active biomass per unit of reaction volume is available. There are also advantages in the postfermentation preparation. In contrast to structurally viscous fermentation broths with pellets, which in industrial processes require a special extraction decanter for being separated off (Muttzall, K., Einführung in die Fermentationstechnik [“Introduction to Fermentation Technology”], Behr's Verlag, Hamburg, 1992), very small cell assemblages or individual cells can be decanted more simply, effectively and thus economically, for instance via filtration units.

Moreover, with this technology, filamentous organisms can be cultivated on an extremely small scale in microtiter plate formats for the purpose of high throughput analysis. For instance, “screening phases” for mutants of interest can be shortened considerably. Moreover, different substances can be tested for specific activities on the cultivated organisms even with a high throughput.

By separating the hyphenes to the point of single cells, a simplified possible way of genetically manipulating these organisms is also achieved. The cultivation method presented here has the effect that the molecular biological transformation of these organisms, for instance, by means of DNA is simplified definitively. In the conventional method, the mycelium must first be mechanically destroyed, which causes a considerable loss of active biomass and thus for instance lowers the transformation efficiency severely. In the cultivation method developed here, the mycelium formation is suppressed, and the cells present are for the most part vital. A drastic increase in the transformation efficiency can therefore be assumed, which is a decisive parameter in molecular biological tasks.

### DESCRIPTION OF THE FIGURES

FIG. 1: Micrograph of the pellet growth of Caldariomyces fumago in a conventional submerged cultivation.

FIG. 2: Micrograph of the growth of Caldariomyces fumago in submerged cultivation when the method of the invention is employed. The aluminum oxide particles used here (indicated by arrows as examples) cause a formation of the fungal mycelium comprising individual hyphenes.

### EXAMPLE 1

The effectiveness of the novel method presented here for submerged cultivation of filamentous organisms will be described in further detail below by means of an example of an experiment:

In this experiment, using the method of the invention, the typical pellet growth of Caldariomyces fumago in the conventional submerged cultivation is for instance to be suppressed. To that end, first, a preculture of the fungus is prepared.

### Preculture

From an agar plate that has been completely overgrown with Caldariomyces fumago, a piece of overgrown agar approximately 1 cm×1 cm in size was stamped out and placed in a 100 ml Erlenmeyer flask. The Erlenmeyer flask was then filled with 30 ml of potato/glucose medium and incubated for approximately two weeks while being shaken at 120 rpm and at 27° C.

### Primary Culture

For performing a cultivation experiment using the particles according to the invention, a primary culture was prepared as follows: A 1000 ml Erlenmeyer flask was filled with 400 ml of growth medium and mixed with 3 g of aluminum oxide (from SERVA Heidelberg/aluminum oxide Alcoa A-350). The batch was autoclaved by heating to 121° C. for 20 minutes at 1 bar of overpressure. After that, this medium was inoculated with 10 ml of the preculture, comminuted beforehand using an Ultra-Turrax (10 seconds at stage 5). The primary culture was then incubated while being shaken at 150 rpm and at 27° C.

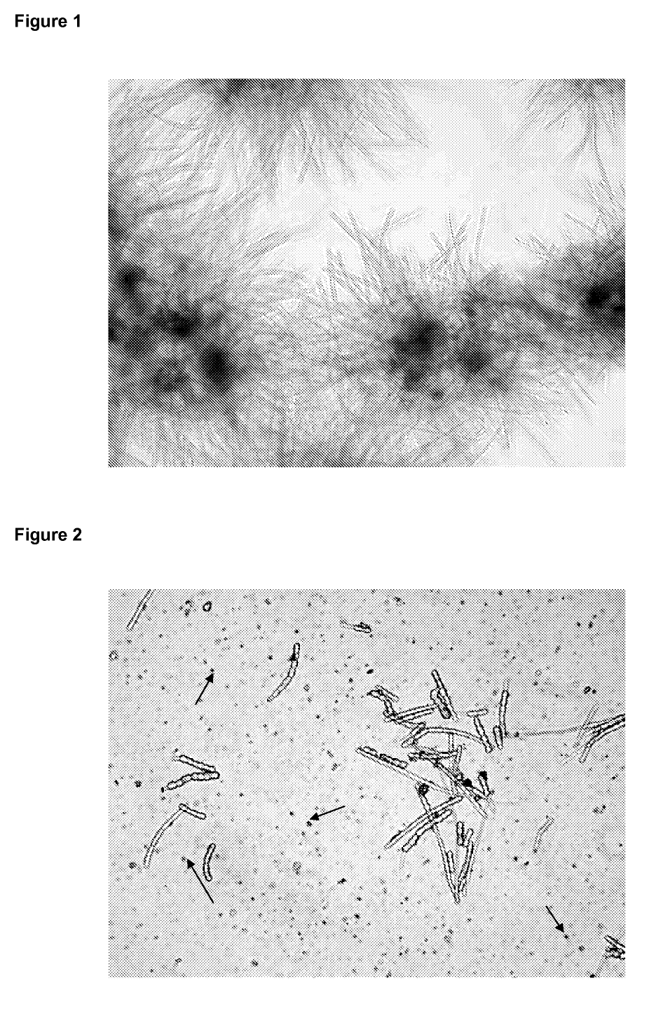

An identical control primary culture, but without the addition of aluminum oxide particles, with Caldariomyces fumago contains the typical pellets as well as microscopically readily apparent cell agglomerates (see FIG. 1).

In contrast to this, micrographs of the primary culture of Caldariomyces fumago in the presence of 0.75% (w/v) of aluminum oxide document the fact that the formation of cell agglomerates does not happen; instead, a formation of the fungal mycelium comprising individual hyphenes can be observed (see FIG. 2).

It can also be found that the metabolic activity of the biomass formed is increased by a factor of 2 to 3 in the presence of aluminum oxide particles. To that end, after cultivation for 12 to 14 days, the quantity of the enzyme chloroperoxidase secreted by Caldariomyces fumago is ascertained. This is done by measuring the specific activity of this enzyme in the culture residue. In control batches without aluminum oxide, this activity is only from 0.45 to 0.75 units/mg of dry biomass, while in the presence of aluminum oxide particles, the figure is approximately 1.5 units/mg of dry biomass.

### Potato/Glucose Medium

Boil 200 g of peeled potatoes per liter of medium in distilled water until mealy;
Press the brew through a cloth in order to retain large pieces of potato;
Fill the “filtrate” with water up to 60% of the final volume;
Dissolve 30 g/L of glucose in the remaining 40% of the water;
Autoclave the two solutions separately for 20 minutes at 121° C. and 1 bar of overpressure;
After the autoclaving, combine the two solutions.

### Growth Medium

| | |
|---|---|
| Glucose | 40 g/L |
| Malt extract | 40 g/L |
| NaNO₃ | 2 g/L |
| KCl | 2 g/L |
| KH₂PO₄ | 2 g/L |
| MgSO₄*7H₂O | 1 g/L |
| FeSO*7H₂O | 0.02 g/L |

Fill with 1000 ml of distilled water and filter in sterile fashion via a 2 μm filter.

### EXAMPLE 2

In the following example of an experiment, the effectiveness of the method of the invention on the growth of Caldariomyces fumago (DSM 1256) during fermentation processes in a bioreactor was tested.

### Preculture

From an agar plate that has been completely overgrown with Caldariomyces fumago, a piece of overgrown agar approximately 1 cm×1 cm in size was stamped out and placed in a 100 ml Erlenmeyer flask. The Erlenmeyer flask was then filled with 30 ml of potato-glucose medium (from Example 1) and incubated for seven days while being shaken at 180 rpm and incubated at 27° C. Next, the culture was comminuted with the aid of an Ultra-Turrax (30 seconds, stage 3).

### Fermentation

In a bioreactor (KLF 2000, Bioengineering) with a capacity of 3.7 L, fermentation was to be done with a total of 2.6 L (fructose-minimal medium). To that end, the bioreactor was filled with 2200 ml of fructose solution (40 g/L) and 10 g/L each of the particles according to the invention comprising aluminum oxide (SERVA, Alcoa A-305) and talcum (Sigma-Aldrich, talc powder, 243604) and then autoclaved for 20 minutes at 121° C. After that, the separately sterile-filtered salt solution of the fermentation medium (300 ml) was added to the bioreactor. Next, the bioreactor was inoculated with 100 ml of homogenized preculture. The agitation speed was 1000 rpm and the fermentation temperature was 28° C. The cultivation lasted 10 days.

In the fermentation broth, in the presence of the particles, the formation of a very fine mycelium occurred, to a marked extent in small agglomerates up to 0.5 mm in diameter, separate, individual hyphenes, and single cells. The use of the method made it possible for the otherwise strong adhesion of mycelium to reactor fixtures as a consequence of the typical filamentous growth of the organism to be extensively suppressed. Because of the optimal rheological conditions, good, uniform mixing of the culture was possible, and as a result, a uniform supply of nutrients could be done. It was thus possible to assure more-precise and replicable process control.

In an identical fermentation of the organism without the addition of the particles, very major adhesion of Caldariomyces fumago to the reactor walls and extreme overgrowth of the reactor fixtures and measuring devices (such as the pH electrode) occurred. In contrast to the fermentation with particles, what was obtained was a heterogeneous, severely clumped culture. The uniform mixing of the culture proved to be from difficult to impossible, because of clumping of different sizes and the adhesion of the organism to the reactor fixtures (see FIG. 1).

### Fructose-Minimal Medium

| | |
|---|---|
| Glucose | 40 g/L |
| NaNO₃ | 2 g/L |
| KCl | 2 g/L |
| KH₂PO₄ | 2 g/L |
| MgSO₄*7H₂O | 1 g/L |
| FeSO*7H₂O | 0.02 g/L |

pH value 6.5.

### EXAMPLE 3

The method of the invention exhibits a marked effect on the morphology of Penicillium digitatum (DSM 62840), which in the submerged cultivation exhibits a growth in round pellets of 5 to 60 mm in diameter.

### Preculture

From an actively growing mother culture in a test tube with oblique agar, using a sterile inoculation eyelet, a small piece of mycelium was scratched off and placed in 50 ml of Penicillium medium in a 100 ml Erlenmeyer flask. After cultivation for two days at 200 rpm on the shaker at room temperature, round pellets formed, with a diameter of 4 to 8 mm. With the aid of an Ultra-Turrax (20 seconds, stage 4), the pellets were mechanically comminuted and the culture was homogenized.

### Primary Culture

In a 300 ml Erlenmeyer flask, 1.5% (w/v) of the particles of the invention (aluminum oxide Alcoa A-350; Serva Heidelberg) were weighed and autoclaved in 5 ml of citrate buffer (100 mM), pH 6.5, for 20 minutes at 121° C. After the addition of 95 ml Penicillium medium, inoculation was done with 1 ml of the preculture. The cultures were then incubated while being shaken at 180 rpm and at room temperature for ten days. An identical control culture with 5 ml of citrate buffer and 95 ml of medium without particles was made in parallel. In this control culture, large pellets grew, ranging in diameter 5 mm to 60 mm. In contrast to that, in the presence of the particles, after ten days markedly smaller pellets grew, with a diameter of from 0.2 to 1.2 mm. In addition, the formation of numerous single hyphenes was observed. It was possible to reduce the pellet size by a factor of up to 60.

### Growth Medium for Penicillium:

| | |
|---|---|
| Glucose | 10 g/L |
| Malt extract | 20 g/L |
| Peptone | 15 g/L |
| Yeast extract | 5 g/L |

A pH value of 6.0 was established before the autoclaving (120° C. for 20 minutes).

### EXAMPLE 4

With Penicillium chrysogenum (DSM 848), it was possible by using the method of the invention to achieve a pronounced reduction in the pellet size.

### Preculture

To the freeze-dried pellet of P. chrysogenum mother culture, 1 ml of Penicillium medium (from Example 3) was added. After an incubation period of 30 minutes at room temperature, the suspension of the prepared culture was stirred with a sterile inoculation loop. Next, 50 ml of Penicillium medium was inoculated with 100 μl of the suspension in a 100 ml Erlenmeyer flask. Cultivation was done for two days on the shaker at 190 rpm and at room temperature.

### Primary Culture

For inoculating the primary cultures, 1.5% (w/v) each of the aluminum oxide (SERVA, Alcoa A-305) and talcum (Sigma-Aldrich, talc powder, 243604) were autoclaved in 5 ml of citrate buffer (100 mM), pH 6.5, in a 300 ml Erlenmeyer flask for 20 minutes at 121° C. After the addition of 95 ml of Penicillium medium, inoculation was done with 1 ml of the two-day-old preculture and incubation was done with shaking at 190 rpm for 6 days. Parallel to this, a control culture comprising 5 ml of citrate buffer and 95 ml of Penicillium medium was inoculated with 1 ml of preculture and cultivated in parallel.

While in the control culture relatively large pellets of 2 to 60 mm grew, the pellet size could be significantly reduced by the addition of the particles. In the presence of aluminum oxide and talcum, very fine cultures almost entirely comprising single-cell meshes with only a few pellets up to 3 mm in size were present.

### EXAMPLE 5

With Emericella nidulans (DSM 820), by the use of the method of the invention, it was possible to achieve a growth comprising single hyphens and to largely prevent the formation of pellets.

### Preculture

50 ml of potato-glucose medium (see Example 1) in a 100 ml Erlenmeyer flask were inoculated with some mycelia from an actively growing oblique agar culture of Emericella nidulans and incubated while being shaken at 180 rpm for 4 days at room temperature.

### Primary Culture

In a 300 ml Erlenmeyer flask, 1.5% (w/v) of talcum particles (Sigma-Aldrich, talc powder, 243604) were weighed and autoclaved in 5 ml of citrate buffer (100 mM), pH 6.5, for 20 minutes at 121° C. After the addition of 95 ml of SNLH medium, inoculation was done with 2 ml of a four-day-old preculture, which had been comminuted beforehand with an Ultra-Turrax (20 seconds, stage 4), and incubation was done with shaking at 190 rpm for 10 days. A control culture without particles was inoculated and incubated in parallel.

After 10 days it could be determined that there was a pronounced morphological difference between the culture containing talcum and the control culture. In the control culture, round pellets with diameters of 1 to 3 mm had formed, while in the presence of the talcum, a very fine, homogeneous culture was present, which virtually entirely comprised single hyphens and meshes comprising single cells. The single cells have lengths of approximately 50 to 200 μm.

### SNLH Medium (Standard Nutrient Solution-Yeast Extract:

| | | |
|---|---|---|
| D-glucose monohydrate | 30 | g/L |
| L-aspartame monohydrate | 4.5 | g/L |
| (L-aspartamic acid monohydrate) | | |
| KH₂PO₄ | 1.5 | g/L |
| MgSO₄ × H₂O | 0.5 | g/L |
| Yeast extract | 3 | g/L |
| Trace element solution | 1 | ml |

### Trace Element Solution:

| | | |
|---|---|---|
| FeCl₃ × 6 H₂O | 80 | mg/L |
| ZnSO₄ × 7 H₂O | 90 | mg/L |
| MnSO₄ × H₂O | 30 | mg/L |
| CuSO₄ × 5 H₂O | 5 | mg/L |
| Titriplex III | 0.4 | g/L |

The pH value of the medium was adjusted to pH 6.0 with 1 N of KOH before autoclaving. All the ingredients of the medium with the exception of the glucose were dissolved in 800 ml of distilled water and autoclaved for 20 minutes at 120° C. The glucose was mixed separately with 200 ml of water and added after the autoclaving to the cooled medium.

### EXAMPLE 6

With Acremonium chrysogenum (DSM 880), it was possible with the method of the invention to achieve a largely filamentous growth.

### Preculture

From an actively growing mother culture on oblique agar, mycelium of A. chrysogenum was removed using a sterile inoculation loop and transferred to a 100 ml Erlenmeyer flask filled with 50 ml of SNLH medium (Example 5). The culture was incubated for 4 days at room temperature on the shaker at 190 rpm.

### Primary Culture

In a 300 ml Erlenmeyer flask, 1.5 g of the aluminum oxide particles (SERVA, Alcoa A-305) and talcum particles (Sigma-Aldrich, talc powder, 243604) were weighed, dissolved in 5 ml of citrate buffer (100 mM), pH 6.5, and autoclaved for 20 minutes at 121° C. After the addition of 95 ml of SNLH medium at pH 6.0, inoculation was done with 2 ml of the four-day-old preculture. In parallel, a control culture comprising 3 ml of citrate buffer and 95 ml of SNLH medium was inoculated accordingly. After 6 days of cultivation on the shaker at 190 rpm at room temperature, the effect of the particles on the fungus morphology was clearly apparent. In the control culture in SNLH medium, round pellets with a diameter of 1 to 10 mm formed, while in the presence of the particles, loose, ramified mycelium meshes with single hyphenes, and very small elliptical hyphen assemblages formed.

In micrographs of the cultures in the presence of the particles, the formation of single cells and the progressive dissolution of pellet structure were clearly apparent.

Besides a pronounced reduction in the pellet size and a change in the morphology, an increase in the dry biomass also occurred, by a factor of 1.6 in the presence of aluminum oxide and by a factor of 1.4 in the presence of talcum.

### EXAMPLE 7

By the use of the cultivation method of the invention, it was successfully possible with Pleurotus sapidus (DSM 8266) as well to prevent the formation of clumplike pellets.

### Preculture

From an agar plate completely overgrown with P. sapidus, a piece of overgrown agar approximately 1 cm×1 cm in was cut out and transferred to a 100 ml Erlenmeyer flask with 50 ml of potato-glucose medium (Example 1). The flask was then incubated, while being shake at 180 rpm, for 1 week at room temperature.

### Primary Culture

For the primary cultures, 300 ml Erlenmeyer flasks were filled with 1.5 g of aluminum oxide (SERVA, Alcoa A-305) and 5 ml of citrate buffer (100 mM), pH 6.5, and autoclaved for 20 minutes at 121° C. After the addition of 95 ml of SNLH medium (Example 5), inoculation was done with 2 ml of a seven-day-old preculture, comminuted beforehand (20 seconds at stage 4) with the Ultra Turrax. In an identically prepared control culture without particles, after 10 days a clump 30 mm in diameter has grown, while in the presence of aluminum oxide, many small pellets of 0.1 to 6 mm and many hyphene assemblages 100 to 300 μm in size have grown. There was also a large number of single hyphenes in the culture.

The addition of particles moreover led to an increase in the dry biomass by a factor of 3.

### EXAMPLE 8

Rhizopus oryzae (DSM 907) exhibits a clumpy growth, which it was possible to suppress by the use of the method according to the invention.

### Preculture

A 100 ml Erlenmeyer flask was filled with 50 ml of potato-glucose medium (from Example 1) and inoculated with mycelium from an R. oryzae oblique agar culture. This was incubated for 3 days at room temperature on a shaker at 190 rpm.

### Primary Culture

In 300 ml Erlenmeyer flasks, 1.5 g of aluminum oxide (SERVA, Alcoa A-305) with 5 ml of citrate buffer (100 mM), pH 6.5, were autoclaved for 20 minutes at 121° C. After the addition of 95 ml of SNLH medium (Example 5), inoculation was done with 2 ml of the preculture homogenized beforehand (30 seconds at stage 4) with the Ultra-Turrax and incubated while being shaken at 190 rpm for 8 days at 25° C. In the control culture prepared in parallel comprising 5 ml of citrate buffer and 95 ml of SNLH medium without particles, a mycelium clump 80 mm in diameter formed, while in the presence of aluminum oxide, many smaller pellets 1 to 5 mm in diameter grew, and there were also meshes comprising single hyphenes. Besides a reduction in the pellet size of up to a factor of 32, an increase in the dry biomass by 46% took place as a result of the addition of aluminum oxide.

### EXAMPLE 9

While Chaetomium globosum (DSM 1962) in conventional submerged culture grows in round pellets up to 5 mm in diameter, by the use of the method of the invention the pellet size is reduced and the formation of single cells is brought about.

### Preculture

To a freeze-dried pellet of C. globosum (DSMZ), 1 ml of SNLH medium (Example 5) was added and incubation was done for 30 minutes at room temperature. 50 ml of SNLH medium in a 100 ml Erlenmeyer flask was inoculated with 50 μl of the prepared, freeze-dried mother culture, and incubation was done for 3 days at room temperature on the shaker at 190 rpm.

### Primary Culture

In a 300 ml Erlenmeyer flask, 5 ml of citrate buffer (100 mM), pH 6.5, with 1.5% (w/v) talcum powder (Sigma-Aldrich, talc powder, 243604) were autoclaved for 20 minutes at 121° C. After the addition of 95 ml of SNLH medium, inoculation was done with 2 ml of the three-day-old preculture and cultivation was done with shaking at 190 rpm for 6 days at room temperature. Parallel to this, a control culture comprising 5 ml of citrate buffer (100 mM), pH 6.5, and 95 ml of SNLH medium was inoculated with 2 ml of the preculture and cultivated.

If the cultures of C. globosum after 5 days of cultivation are compared, a pronounced difference in the pellet size and morphology can be seen. While in the control culture, round pellets with diameters of 1 to 5 mm have grown, in there presence of the talcum particles there are markedly smaller pellets, 0.2 to mm in size, as wells as numerous single cells. The addition of talcum to the growth medium brought about a reduction in the pellet size by a factor of up to 25.

### EXAMPLE 10

The filamentous bacterium Streptomyces aureofaciens (DSM 40127) in submerged culture forms pellets whose size it was possible to reduce by the use of the method of the invention.

### Preculture

First, a preculture of S. aureofaciens is prepared. For that purpose, first, to the freeze-dried mother culture, 1 ml of the recommended growth medium (Medium 65, DSMZ) is added. After an incubation time of 30 minutes at room temperature, the cell suspension is stirred with a sterile inoculation loop. Next, 50 μl of the prepared mother culture in 40 ml of growth medium (Medium 65, DSMZ) were transferred to a 100 ml Erlenmeyer flask. The thus-inoculated culture was incubated on a shaker at 190 rpm for 2 days at room temperature.

### Primary Culture

In two 300 ml Erlenmeyer flasks, 1.5% (w/v) of aluminum oxide (SERVA, Alcoa A-305) and talcum (Sigma-Aldrich, talc powder, 243604) were weighed and autoclaved with 5 ml of citrate buffer (100 mM), pH 6.5 for 20 minutes at 121° C. After the addition of 95 ml of growth medium, inoculation was done with 1 ml of the two-day-old preculture and cultivation was done for 5 days at 190 rpm on the shaker. In parallel, a control culture without particles with 5 ml of citrate buffer and 95 ml of grown medium was incubated.

The particles exhibit an effect on the pellet size of t.cm. While the pellets in the control culture reached sizes of 900 to 1200 μm, the pellets that had grown in the presence of the aluminum oxide particles had a diameter of 75 to 350 μl and those that had grown in the presence of talcum particles had a size of 60 to 500 μm. The formation of single cells was also favored; in the cultures with particles, compared to the control culture, there were larger quantities of single cells.

### Medium 65

| | |
|---|---|
| Glucose | 4 g/L |
| Yeast | 4 g/L |
| Malt extract | 10 g/L |

Adjust pH value to 7.2.

1. A method for submerged cultivation of filamentous fungi or bacteria that are mycelial or that grow in multi-cell assemblages, wherein the development of cell agglomerates, mycelial assemblages and pellets as well as the tendency to adhere to abiotic surfaces during the cultivation are reduced or prevented,
in that the cultivation is performed in the presence of particles, having a size of up to several millimeters, that are insoluble or only partly soluble in the cultivation fluid;
and the particles comprise metal, metal oxide, glass, plastic, carbon, crystalline salt, a semimetal, or mixtures of these materials; and either
the cultivation serves the purpose of biotechnological extraction of proteins; or
following the cultivation, biocatalytic processes are performed with cells that do not grow or that grow only poorly.

2. (canceled)

3. (canceled)

4. (canceled)

5. The method as defined by claim 1, wherein the cultivated organism is the fungus Caldariomyces fumago.

6. The method as defined by claim 1, characterized in that the cultivated organism is bacteria of the genus Streptomyces.

7. The method as defined by claim 1 wherein the particles comprise aluminum oxide.

8. The method as defined by claim 1 wherein the insoluble particles comprise silicate particles or silicate splinters in the form of glass.

9. (canceled)

10. (canceled)

11. The method as defined by claim 1 wherein the cultivation of the filamentous fungi and the bacteria that are mycelial or that grow in multi-cell assemblages is conducted for the purpose of high throughput analysis.

12. (canceled)

13. The method as defined by claim 1 wherein the method enables, facilitates or increases the efficiency of the genetic manipulation of foreign or endogenous nucleic acid.

14. The method as defined by claim 2 wherein the particles comprise aluminum oxide.

15. The method as defined by claim 3 wherein the particles comprise aluminum oxide.

16. The method as defined by claim 2 wherein the insoluble particles comprise silicate particles or silicate splinters in the form of glass.

17. The method as defined by claim 3 wherein the insoluble particles comprise silicate particles or silicate splinters in the form of glass.

18. The method as defined by claim 2 wherein the cultivation of the filamentous fungi and the bacteria that are mycelial or that grow in multi-cell assemblages is conducted for the purpose of high throughput analysis.

19. The method as defined by claim 3 wherein the cultivation of the filamentous fungi and the bacteria that are mycelial or that grow in multi-cell assemblages is conducted for the purpose of high throughput analysis.

20. The method as defined by claim 4 wherein the cultivation of the filamentous fungi and the bacteria that are mycelial or that grow in multi-cell assemblages is conducted for the purpose of high throughput analysis.

21. The method as defined by claim 5 wherein the cultivation of the filamentous fungi and the bacteria that are mycelial or that grow in multi-cell assemblages is conducted for the purpose of high throughput analysis.

22. The method as defined by claim 14 wherein the cultivation of the filamentous fungi and the bacteria that are mycelial or that grow in multi-cell assemblages is conducted for the purpose of high throughput analysis.

23. The method as defined by claim 15 wherein the cultivation of the filamentous fungi and the bacteria that are mycelial or that grow in multi-cell assemblages is conducted for the purpose of high throughput analysis.

24. The method as defined by claim 16 wherein the cultivation of the filamentous fungi and the bacteria that are mycelial or that grow in multi-cell assemblages is conducted for the purpose of high throughput analysis.

25. The method as defined by claim 17 wherein the cultivation of the filamentous fungi and the bacteria that are mycelial or that grow in multi-cell assemblages is conducted for the purpose of high throughput analysis.

26. The method as defined by claim 2 wherein the method enables, facilitates or increases the efficiency of the genetic manipulation of foreign or endogenous nucleic acid.
